(19) United States
(12) Patent Application Publication
(10) Pub. No.: US 2020/0275837 A1
(43) Pub. Date: Sep. 3, 2020

(54) Treatment of substance abuse and addictive disorders

(71) Applicant: Alexander Vladimir Astafiev, Las Vegas, NV (US)

(72) Inventor: Alexander Vladimir Astafiev, Las Vegas, NV (US)

(21) Appl. No.: 16/261,568

(22) Filed: Jan. 30, 2019

(51) Int. Cl. A61B 5/00; A61N 7/02; A61B 5/055; A61B 5/01; G01R 33/48

(52) U.S. Cl. CPC A61B 5/0036; A61N 7/02; A61B 5/055; A61N 2007/003; A61B 5/0042; G01R 33/4806; A61B 5/015

(57) ABSTRACT

Treatment of substance abuse and addictive disorders by non-invasive transcranial brain high-intensity focused ultrasound guided and controlled by magnetic resonance imaging or by transcranial non-invasive MRI-guided focused ultrasound surgery. The majority of pathological zones associated with addictions are located in the anterior cingulate cortex, a part of the prefrontal cortex. Localization of the pathological zone will be performed by functional Magnetic Resonance Imaging (fMRI) and Diffusion Tensor imaging (DTI) techniques. The therapy is designed to mitigate or even destroy pathological neuron networks. Absorption of ultrasound waves in the selected focal area will heat and consequently thermally destroy the pathological zone. MR imaging and MR-thermometry allows real-time therapy guidance and ablate targeted tissue without damaging normal structures.

### CROSS-REFERENCE TO RELATED APPLICATIONS

Provisional patent application No.: U.S. 62/625,109; filled Feb. 1, 2018

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

### (1) Field of the Invention:

The present invention relates to methods of treating substance abuse and addictive disorders.

### (2) Description of Related Art including information disclosed under 37 CFR 1.97 and 1.98

Substance abuse and addictions are chronic brain diseases with sociocultural, genetic, and neurodevelopmental components. Initiation of drug use occurs most often during adolescence and early adulthood.

Vulnerability to drug abuse is related to both reward seeking and impulsivity, two constructs thought to have a biological basis in the prefrontal cortex (PFC). These conditions create pathological neurons networks, weakening self-regulation, and increasing dysphoria, and negative mood. That results in forming pathological zones “pleasure centers”. Drug-induced impairments are long lasting and produce pathological reward circuits on the reward pathway in the brain. The reward pathway involves several parts of the brain: the prefrontal cortex, the ventral tegmental area (VTA), and the nucleus accumbens.

Stereotactic cryodestruction of pathological zones in prefrontal cortex of human brain demonstrate high success rate in treatment of drug abuse. The first surgeries were performed in Russia and later continued in China. This high risk of invasive surgeries was banned for medical practice due to lack data on long term outcome and growing outrage over ethical issues. Now some doctors in these countries have been allowed to perform the procedure for research purposes.

Now the proposed methods to ablate brain tissue with high-intensity focused ultrasound (HIFU). These technologies have been suggested for treatment of Parkinson's disease, epilepsy, tumors and to inhibit transmission of nerve signals in the brain.

### BRIEF SUMMARY OF THE INVENTION

Treatment of substance abuse and addictive disorders by non-invasive transcranial-brain high-intensity focused ultrasound (HIFU) guided and controlled by magnetic resonance imaging (MRI) or by transcranial non-invasive MRI-guided focused ultrasound surgery (MRgFUS)

### BRIEF DESCRIPTION THE SEVERAL VIEWS OF THE DROWING

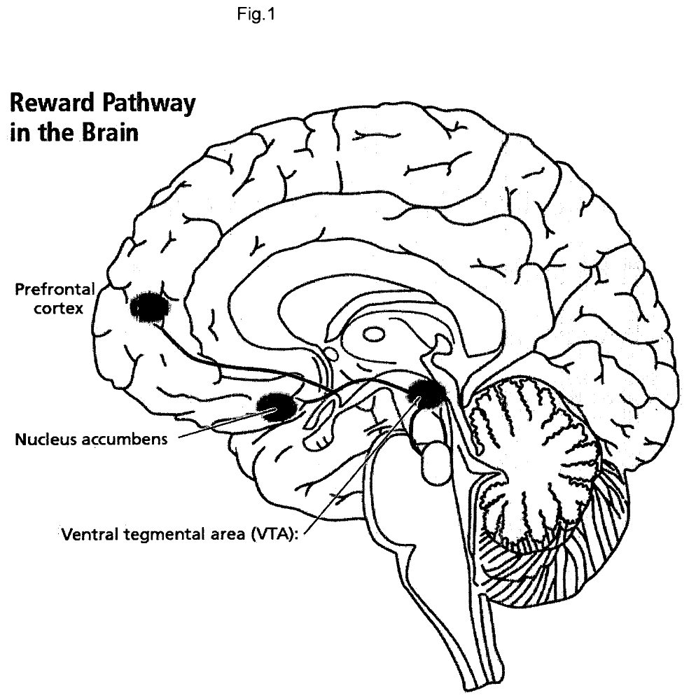

FIG. 1 shows. The reward pathway involves several parts of the brain: the prefrontal cortex, the nucleus accumbens, and the ventral tegmental area (VTA).

The majority of pathological zones associated with addictions are located in the prefrontal cortex.

### DETAILED DESCRIPTION OF THE INVENTION

Therapy designed to mitigate or even destroy pathological neuron networks will be beneficial for the treatment of addiction.

MRI-guided focused ultrasound surgery (MRgFUS) and MRI guided high-intensity focused ultrasound (MR-HIFU) are vendors' specific terminology and in many cases are synonyms.

Philips Healthcare developed MR-HIFU and General Electric with cooperation of InSightec Ltd developed MRgFUS.

MR HIFU has a wider meaning and includes all biological effects of focused ultrasound. MRgFUS uses only thermal and cavitation effects which produces ablation of brain tissue.

Integrating HIFU and MRI allows medical providers to localize, target, and monitor treatment of the pathological zone in the patient's brain.

Treatment process can be divided in two stages:

First step is localization of the pathological zone by functional Magnetic Resonance Imaging (fMRI) and Diffusion Tensor imaging (DTI) technics.

Addictions are chronic brain diseases with sociocultural, genetic, and neurodevelopmental components. These conditions create pathological neurons networks, weakening self-regulation, and increasing dysphoria, and negative mood. Drug-induced impairments are long lasting and produce pathological reward circuits on the reward pathway in the brain. The reward pathway involves several parts of the brain: the prefrontal cortex, the ventral tegmental area (VTA), and the nucleus accumbens.

The anterior cingulate cortex (ACC) is a part of the prefrontal cortex and a cingulate cortex resembling a “collar” surrounding the frontal part of the corpus callosum. The majority of pathological zones associated with addictions are located in this area.

Destruction pathological zone in this brain area eliminates psychological dependence, and with a combination with detoxication statistically shows 65% recovery rate. It is the highest success rate currently.

The next step is concentration of ultrasound (US) waves to a focal point. Non-invasive intracranial application of US on the targeted zone needs to use ultrasound phased arrays for correction of distortion produced by skull bones.

Due to US absorption, the selected focal area will be heated and consequently thermally destroyed. Cell death may occur due to acoustic cavitation, and if the oscillating bubble disrupts the cell membrane.

MR imaging and MR-thermometry allows real-time therapy guidance. It also establishes control localization and temperature in the selected pathological zone to ablate targeted tissue without damaging normal structures. The spatial accuracy of the non-invasive procedure and the sharp delineation of the induced tissue lesions have led to the term “focused ultrasound surgery”(FUS).

This precision makes MRgFUS an attractive alternative to surgical resection, cryodestruction; or radiation therapy

In addition to thermal ablation (FUS), ultrasound waves can temporarily change vascular or neurons membrane permeability. In the brain, ultrasound is used to open the blood brain barriers for drug administration, release or activate various compounds and change gene activity. These affects may allow treat addiction without neurons destruction. This treatment approach is requiring additional research.

1. Treatment of substance abuse and addictive disorders

2. The method of claim 1 wherein treatment by non-invasive transcranial brain high-intensity focused ultrasound (HIFU)

3. The method of claim 1 wherein treatment by transcranial non-invasiveMRI-guided focused ultrasound surgery (MRgFUS)

4. The method of claim 1 wherein guided by magnetic resonance imaging (MRI)

5. The method of claim 1 wherein controlled by magnetic resonance imaging (MRI)

6. The method of claim 1 wherein control temperature in the selected pathological zone by MR-thermometry during treatment

7. Localization of the pathological zone by Magnetic Resonance Imaging (MRI)

8. The method of claim 7 wherein localization of the pathological zone by functional Magnetic Resonance Imaging (fMRI)

9. The method of claim 7 wherein localization of the pathological zone by Diffusion Tensor imaging (DTI) technique
